(19) United States
(12) Patent Application Publication
(10) Pub. No.: US 2021/0161421 A1
(43) Pub. Date: Jun. 3, 2021

(54) BIOIMPEDANCE MEASUREMENT DEVICE

(71) Applicant: SENTINHEALTH, Paris (FR)

(72) Inventors: Jean-Marc BOUREZ, Paris (FR); Cindy DOPIERALA, Saint Mury Monteymond (FR)

(73) Assignee: SENTINHEALTH, Paris (FR)

(21) Appl. No.: 17/048,435

(22) PCT Filed: Apr. 17, 2019
(86) PCT No.: PCT/FR2019/050923

§ 371 Date: Oct. 16, 2020

(30) Foreign Application Priority Data
Apr. 18, 2018 (FR) 18 53387

(51) Int. Cl. A61B 5/053; G01N 27/02; A61B 5/00

(52) U.S. Cl. CPC A61B 5/053; G01N 27/028; A61B 5/0537; A61B 5/684; A61B 5/7278; A61B 5/681

(57) ABSTRACT

A bioimpedance-measuring device comprising a band configured so as to surround a wrist of a first arm of a user and on which there are arranged at least two current-injecting electrodes and at least two potential-measuring electrodes, said measuring device furthermore comprising a system for collecting raw bioimpedance data from the potential-measuring electrodes and a system for processing raw data in order to obtain at least one monitoring measurement and information signifying a pulmonary bioimpedance condition, said collection system being carried by the band and configured so as to communicate with the processing system.

### TECHNICAL FIELD

The field of the present invention is that of measuring devices applied to bodily components, more particularly devices capable of measuring the bioimpedance of a human body.

### BACKGROUND ART

As is known, heart failure in humans may cause fluid to build up in the lungs. This aqueous buildup, otherwise called edema, impairs correct respiration: lodged in the lung tissue at the extracellular level, water blocks the gaseous exchanges of oxygen and carbon dioxide that usually take place there.

Cardiac decompensation may thus lead to cardiogenic pulmonary edema. Breathlessness and shortness of breath are among the symptoms induced by respiratory compensation, increasing the respiratory work of the lungs and in particular causing chest pain. These symptoms worsen as far as major respiratory distress if the buildup of fluid is not detected in advance. Pulmonary edema is considered in the medical context to be a vital emergency that must be treated at the first signs of onset, the treatments being all the more severe the later the diagnosis, the tissue being less engorged at an initial stage.

Any patient at risk of cardiac pathology should therefore be vigilant because of the possible onset of pulmonary edema. Patients at risk are generally monitored through regular medical examinations, such as osculation by the practitioner, chest radiography, blood tests and/or electrocardiograms in order to identify cardiac arrhythmias. In terms of prevention, the patient is forced to closely monitor his lifestyle and to treat his cardiac pathology in order to avoid underlying complications of pulmonary edema.

However, medical monitoring remains restrictive for the patient, since he depends on the medical community and on the practitioner to carry out an assessment of his pulmonary and cardiac condition. Another drawback lies in the regularity of the monitoring, the patient having to frequently submit to the medical community in order to prevent aggravated complications. Moreover, monitoring cannot reasonably be carried out on a long-term multi-day basis for all patients, in particular those who maintain good autonomy.

A measurement, by the patient himself, of a variation in the volume of fluid in lung tissue would make it possible to assess the onset of edema with relative autonomy. The bioimpedance measurement is a non-invasive measurement providing information in particular about a liquid composition of the body, such as the presence of edema. Known bioimpedance-measuring devices do not however make it possible to specifically and practically assess the presence of thoracic edema, and more specifically of pulmonary edema.

By way of example, and as known for example from documents US2016089053 or US2018000375, these bioimpedance-measuring devices are used to determine a body composition of the user, and for example a level of fat present in the body.

### SUMMARY OF THE INVENTION

The aim of the present invention is therefore to overcome the drawbacks described above by designing a bioimpedance-measuring device able to be used easily and repeatedly by the patient to monitor his pulmonary condition.

One subject of the invention is therefore a bioimpedance-measuring device, characterized in that it comprises a band configured so as to surround a wrist of a first arm of a user and on which there are arranged at least two current-injecting electrodes and at least two potential-measuring electrodes, a first current-injecting electrode and a first potential-measuring electrode being arranged on an inner surface of the band and configured so as to be in contact with the wrist, a second current-injecting electrode and a second potential-measuring electrode being arranged on an outer surface of the band and each configured so as to have an interface surface with part of the second arm of the user, the measuring device furthermore comprising a system for collecting raw bioimpedance data from the potential-measuring electrodes, and a raw data processing system for obtaining at least one monitoring measurement and information signifying a pulmonary bioimpedance condition, said collection system being carried by the band and configured so as to communicate with the processing system.

The measuring device according to the invention comprises a band worn on the wrist of its user. This band is in contact with the wrist during the measurement. This contact with the wrist is made on the side of the inner surface of the band, at the first current-injecting electrode and at the first measuring electrode. The first current-injecting electrode and the first measuring electrode are thus in contact with the wrist during the measurement. In particular, the band is loose in relation to the wrist. Thus, when no measurement operation is planned, the band is not in close contact with the wrist, so as to avoid a sweating effect that could disrupt future measurements. In order to close a current flow loop necessary for the bioimpedance measurement, the user puts his hand or any part of his arm, which is not equipped with the band, in contact with the second current-injecting electrode and the second measuring electrode. This flow loop is moreover closed only when the first current-injecting electrode and the first measuring electrode are also in contact with the user's skin. Closing the current flow loop and generating a vector capable of performing the bioimpedance measurement is therefore brought about through a positive action by the user.

Advantageously, the total number of electrodes is four: two current-injecting electrodes and two measuring electrodes. The number of four electrodes, although not limiting, makes it possible to optimize the size of the band. Moreover, a four-point measurement makes it possible to minimize the contact impedance caused by the electrodes. It will however be understood that it will be possible to propose, without departing from the context of the invention, a band with a different number of electrodes, provided that the closure of a current flow loop is generated through a positive action by the user, as explained above.

A low-intensity electric current flows between the current-injecting electrodes while passing through the measuring electrodes. Low intensity is understood to mean an intensity less than one milliampere, of the order of 10 to 100 microamperes for a frequency of approximately 10 kHz. The measuring electrodes are arranged on the path of this electric current so as to define an electrical potential value at a given point. The signal seen by the measuring electrodes is an electrical potential that is characteristic, for a current of constant intensity, of an impedance value, in accordance with Ohm's law. The electrical potential resulting from the passage of the current is modified on the basis of the resistance of the biological tissue encountered, and the potential-measuring electrodes arranged on the current path make it possible to quantify the variation in this potential and therefore the variation in bioimpedance for a current of constant intensity.

According to one feature of the invention, the electrodes are distributed into two groups that are arranged, respectively, on the inner face and the outer face of the band, each group comprising a current-injecting electrode and a measuring electrode. The arrangement of each of these groups of electrodes is also different, in order to make it possible, in each group, to position the electrical potential-measuring electrode on the path of the electric current flowing through the body of the user from or to the injecting electrode. More particularly, the first current-injecting electrode and the first potential-measuring electrode of a first group of electrodes are aligned in a direction different from, advantageously perpendicular to, the direction in which the second current-injecting electrode and the second potential-measuring electrode of the second group of electrodes extend.

On each surface of the band, the electrode proximal to a transthoracic segment of the user is the measuring electrode, whereas the electrode distal to this transthoracic segment is the injecting electrode.

By way of example, the first group of electrodes arranged on the inner face, and therefore brought into contact with the arm wearing the measuring device, is configured such that the electrodes are aligned in a first direction along the axis of the arm, with the first injecting electrode arranged on the side of the hand at the end of the arm wearing the measuring device and the first measuring electrode arranged on the side of the user's torso. The group of electrodes arranged on the outer face, and therefore brought into contact with the free hand that closes the loop, is also configured such that the electrodes are aligned in a second direction different from, and in particular substantially perpendicular to, the first direction. The injecting electrode arranged on the outer face of the band may be arranged so as to be covered by the fingers of the free hand that closes the loop, and the measuring electrode is then arranged so as to be covered by the palm of this same hand.

The raw data measured at the measuring electrodes correspond to a potential difference between the two measuring electrodes. These raw data comprise the transthoracic bioimpedance value, but also bioimpedance values of other tissue passed through by the current (muscle tissue, bone tissue, fatty tissue, the vascular system and other organs), and stray impedance values (those of the electrodes for example). It is necessary to take repeated and averaged measurements to identify any variations relating to an evolution of the transthoracic bioimpedance, by comparing a raw data average with a benchmark value. The benchmark value is obtained during a calibration protocol that is described elsewhere.

The set of measurements for obtaining a raw data average is carried out under conditions equivalent to those defined according to the calibration protocol, such that the measurement is not impacted for example by an inappropriate position of the user.

In one exemplary embodiment, the measurement may be replicated several times, within a very short time interval, so as to increase the amount of raw data recovered in order to obtain the average value that is used for comparison with the benchmark value and thus increase the statistical reliability of this set of measurements, without this being too detrimental in terms of the time spent by the user. The impact of cardiac, respiratory, postural and/or ambient noise, which may be estimated to be smoothed over an average obtained from a sufficient amount of raw data, is thus limited, and therefore the variation identified between an average of measured raw data and a benchmark value may be equated to resulting from a variation in the volume of fluid in lung tissue. In one exemplary embodiment, the measurement is acquired by continuously closing the flow loop for an optimized period of time. Optimizing the period of time consists in providing a measurement time that is long enough to filter high-frequency noise, such as cardiac and/or ventilatory noise occurring during this period of time, on the one hand, and that is not too long so as not to make the measurement painful for the user, who has to remain still during this measurement, on the other hand. Advantageously, the measurement is taken during an acquisition of between thirty (30) seconds and forty-five (45) seconds.

Conversion of raw data into useful data is made possible by the processing system. The processing system applies an algorithm for averaging the raw data in order to identify variations in the transthoracic bioimpedance measurement.

By virtue of measurements that are repeated, for example over a day, and averaged, the evolution of the transthoracic bioimpedance value is therefore monitored. Pulmonary edema may be identified when the value of a raw data average, indicative of a transthoracic bioimpedance value, is less than a certain floor value. Floor value is understood to mean a value defining a critical threshold when the value resulting from the measurement of the raw data is lower than this floor value, indicating a degraded physiological state.

This floor value is defined on the basis of a basal state of the user that gave rise, during a calibration process, to the estimation of a reference benchmark value. The floor value corresponds to a value that has dropped by the order of 0.25 to 2.5%, and more particularly by the order of 0.5 to 1%, in comparison with the reference benchmark value, and obtaining a measured value average below that of the floor value is an indicator of a state of decompensation resulting from the buildup of fluid in the chest. Preferred measuring instruments for implementing the measuring device according to the invention will have a resolution capable of detecting this drop. It will be understood that the greater the sensitivity of the instruments that are used, the more variations close to 0.25% with respect to the reference benchmark value will be able to be detected, and the earlier the detection of the state of decompensation will be.

In other words, according to the invention, variations between a benchmark value and each raw data average, as a whole, are looked at in order to determine the relevance of the observed differences. In particular, the inventors have been able to observe that a variation of between 0.25% to 2.5% between averaged raw data and a reference benchmark value is a reliable indicator of a state of decompensation resulting from the buildup of fluid in the chest.

For a raw data average less than this floor value defined by this variation of the order of 0.25 to 2.5% and indicative of edema, a link may be made with heart failure, and the patient should then be taken into medical care.

The flow loop is defined between the wrist of the first arm of the user and the hand or any other part of the second arm, such that it passes through two arms and a transthoracic segment. One advantage of using such a flow loop is that this flow loop is long, passing right through the transthoracic segment between the two arms. In this way, the flow loop makes it possible to define a significant Piccoli vector, for better consideration of the transthoracic segment. It should be noted that the sensitivity of the measurement also depends on the electronic system that is used. The smallest impedance measurements that are taken may be of the order of 10 ohms. This is the case when using an AD5934 chip electrical system, designed to manage the injection of the current and the reception of the signal.

The benefit of a band lies in the fact that it may be worn permanently by the user. The first current-injecting electrode and the first measuring electrode are configured so as to be in contact with the wrist during measuring operations. This contact with the wrist is ensured in particular when the measurement is performed by pressing the other hand or arm against the band, which has the effect of pressing the electrodes against the arm. Contact between the electrodes and the wrist is thus ensured for each measurement. As a result, the band, carrying a miniaturized system, is portable and mobile, limiting the usage constraints for the user. Use thereof for obtaining information relating to the transthoracic bioimpedance is moreover simplified, since it requires only contact-based closure of the flow loop.

The raw data are obtained when the user closes the current flow loop, by touching the outer surface of the measuring device according to the invention. The flow loop is thus defined by the transthoracic segment, the pulmonary assembly and the two arms. “Non-pulmonary impedance values” are understood to mean the set formed by the non-pulmonary bioimpedance values and the stray impedance values induced by the user and inherent to the measuring device according to the invention.

The non-pulmonary bioimpedance corresponds, without limitation, to the bioimpedance values inherent to muscle tissue, bone tissue, fatty tissue, the vascular system and other organs. In other words, the non-pulmonary bioimpedance may correspond to the bioimpedance of the arms and the thorax, excluding the lungs.

The stray impedance values correspond, without limitation, to the impedance values of the electrodes, to movement artefacts (primarily of the arms of the patient), and to the impedance values of the ventilatory and cardiac components. By way of indication, the impedance value of an electrode is between 500 and 1000 ohms. This includes an impedance value due to the electrode itself, a skin/electrode contact impedance value and an impedance value due to skin resistance.

By way of indication, the raw data integrating the pulmonary impedance values and the non-pulmonary impedance values have an estimated value of between 1500 and 2500 ohms.

As has been able to be explained above, according to one aspect of the invention, the raw data are collected over a given period of time, and are then averaged in order to give reliable information on the pulmonary bioimpedance of the user over this given period of time, for example a day. Thus, in order to deduce a result indicative of a specifically pulmonary bioimpedance value from the raw data, the algorithm averages the collected raw data so as to provide a value corresponding to a monitoring measurement. Obtaining an average allows temporal filtering through statistical processing.

According to one aspect of the invention, the processing system is configured so as to determine a deviation between a reference benchmark value and at least one monitoring measurement and to generate a notification for the user when the monitoring measurement has a value less than the value corresponding to the benchmark value and when this deviation is between 0.25% and 2.5% of said value corresponding to the reference benchmark value.

Advantageously, the raw data collected over 24 hours make it possible to establish a monitoring measurement on the basis of a daily average measurement. These daily average measurements are compared with one another in order to detect a deviation from a reference benchmark value obtained through measurements taken over a reference period during the calibration procedure. When the daily average measurements identify values less than the benchmark value, with a deviation of the order of 0.25 to 2.5%, more particularly 0.5 to 1% with respect to the benchmark value, an alert action is taken. It is understood that what is targeted is both a deviation large enough to be indicative of pulmonary edema, which explains why the threshold should be at least 0.25%, and a deviation that is not excessively large, so as to ensure that the detection of a risk of pulmonary edema is achieved within sufficient time to be medically effective.

According to one aspect of the invention, the deviation according to the invention, between 0.25 and 2.5% of the benchmark value, may correspond to a deviation in values of between 5 and 50 ohms. When the deviation measured by the processing system between the benchmark value and a monitoring measurement, obtained through the raw data average, is within these ranges of values, an alert indicating this deviation is notified. This notification is transmitted to the user, for example. It may also be transmitted directly to a center for monitoring the user, for example a medical structure, or to any person authorized to monitor the user. Advantageously, the notification is transmitted to the user and to a person authorized to monitor said user.

The measuring device according to the invention is beneficial in the comparison of a benchmark value, established in order to calibrate the measuring device, and a monitoring measurement, obtained by averaging measurements in order to evaluate the condition of the user. The user is informed of any notable difference. It should be noted that, according to the invention, monitoring measurement is understood to mean a raw data average, for example carried out on raw data recorded over one day. The deviation may be notified for example on the processing system, or on a user interface, as will be described later on.

In order to calibrate the measuring device, the calibration protocol is followed by the user. The user of the measuring device according to the invention must be the person on whom the calibration protocol is carried out. Specifically, the measurements relate to the user, characterizing said user personally. This calibration protocol makes it possible to obtain the benchmark value as defined above, that is to say a reference average obtained from several reference measurements. This makes it possible to establish a baseline of the patient, for example through reference measurements repeated several times a day and/or over several days and averaged.

The calibration protocol determines the protocol according to which the user will have to use the measuring device according to the invention. Advantageously, the calibration protocol is carried out on a user at rest, in a seated position. The monitoring measurements are then carried out systematically in this same context. Compliance with this protocol makes the measurement more reliable.

This calibration protocol should be established before initiating the monitoring measurements. It may also be applied in order to update the measuring device, for example following any modification affecting its use or affecting the user.

Several monitoring measurements are taken at regular intervals in order to be able to detect the onset of pulmonary edema. As has been able to be explained above, a deviation is considered to be significant when the monitoring measurement, corresponding to the raw data average over a for example daily period, has a value less than a floor value corresponding to 0.25 to 2.5%, more particularly 0.5 to 1% of the reference benchmark value. When such a significant deviation is determined by the processing system, the invention may make provision for it to be necessary to confirm the existence of this significant deviation so that the user is not misled by an erroneous measurement. The feasibility of this confirmation depends on the variability and the measurement frequency acceptable to the user.

According to one aspect of the invention, the processing system compares the raw data with at least one limit value. This limit value may for example represent 150% of the benchmark value. An item of raw data with a value greater than this limit value indicates for example that the measurement is incorrect and should be performed again. One cause of an incorrect measurement is for example non-compliance with the contact conditions, that is to say the contact between the wrist of the first arm and the first current-injecting electrode, the wrist of the first arm and the first potential-measuring electrode, the hand part of the second arm and the second current-injecting electrode, the hand part of the second arm and the second potential-measuring electrode, is incorrect. Implementing a limit value is one way of making the measurement more reliable.

The measuring device according to the invention may moreover be coupled to a one-off alert system. This alert system is activated only after a measurement has been taken when the value of the raw data is above the limit value. The user is then warned to reiterate a measurement operation in order to obtain a new value. The alert system may for example correspond to a visual, acoustic or tactile alert. Without limitation, mention may be made of: an indicator light, or a particular display on the user interface, a ringtone, a vibration of the measuring device, or a combination of these examples. The alert may be interrupted manually, or automatically when the measurement is reiterated.

According to one aspect of the invention, the processing system is integrated into the band. Internalizing the processing system in the band is beneficial in terms of the compactness of the measuring device, which is then miniaturized. The raw data are processed directly by the band, the system for collecting the raw data from the potential-measuring electrodes interacting physically with the processing system, for example within the same printed circuit.

As an alternative, the processing system may be externalized. The processing system may then take the form of an independent processing system, or one installed on a medium such as a computer, or any other medium able to contain and carry out the operations of the algorithm specific to the processing device. It will be understood that this externalized processing system is associated with external communication means that are configured so as to communicate with communication means embedded in the band in order to allow raw data to be transmitted between the collection system and the processing system.

The processing system communicates a result indicative of transthoracic or pulmonary bioimpedance to the user. This result is displayed on the processing system directly, or on its medium. The display alternatively takes place on the band, via a user interface for example. This user interface may be an electronic display device, such as a screen. It will be understood that the result may be communicated by the processing system via the abovementioned communication means if the processing system is externalized.

Various communication technologies may be used to connect the processing system to the band and allow the raw data and the calculated result to be transmitted. By way of example, mention may be made of: wireless communication technologies, wave-based communication technologies, such as a technology using Bluetooth, Wi-Fi, or Li-Fi, or wired connection-based technologies.

According to one aspect of the invention, the injecting electrodes are driven so as to use a multi-frequency current. In other words, the current delivered by the electric power supply system to the first current-injecting electrode and to the second current-injecting electrode has a variable frequency. The multi-frequency current makes it possible to measure an extracellular impedance that is inaccessible using a single-frequency method. The extracellular compartment is specifically the one impacted by edema. The robustness of the measurement is thus increased using this approach.

As an alternative, the current may be single-frequency. This frequency is in particular between 5 kHz and 100 kHz. Advantageously, when the power supply system allows delivery of current at different frequencies, the highest frequency is chosen. A high frequency makes it possible to minimize stray impedance values. The stray impedance values are in particular movement artefacts, which may be greater in a measuring device according to the invention, since it is worn on the user's wrist. Choosing a high frequency thus constitutes an electrical optimization for the measuring device according to the invention. A measuring device dispensing a single-frequency current is moreover advantageous by virtue of its simplicity and its low implementation cost.

According to one aspect of the invention, the band is formed by an elastic cylindrical sleeve. Cylindrical sleeve is understood to mean an envelope intended to surround the wrist. This envelope is formed in one piece in the manner of a sheath having a certain elasticity and able to be removed through deformation.

The cylindrical sleeve has a dimension suitable for the wrist. Specifically, the sleeve should be in close physical contact with the user's wrist during the measurement: it should not be too loose, but loose enough not to cause sweating and to avoid prolonged contact of the electrodes with the user's wrist when no measurement is desired. The contact imposed between the wrist and the first current-injecting electrode, and the wrist and the first potential-measuring electrode, both located on the inner surface of the band, imposes its ergonomics on the sleeve. The sleeve should have a circumference close to a circumference of the user's wrist, so as to combine support, ease of installation and conditions suitable for the measurement. These dimensions remain dependent on the material used to produce the sleeve and on its characteristics of its elastic nature.

As an alternative, the band is provided with overlapping ends. The band then has a form close to that of a watch strap, provided with a clasp that makes the overlapping ends interact. When the ends interact, the band forms a closed cylinder. The clasp is ideally adjustable to the dimensions of the wrist. Mention may be made, by way of non-limiting example, of: a clasp with textile hooks and loops, a magnetic clasp, a pin-buckle clasp, an articulated clasp with a folding loop, a snap-lock clasp.

The first current-injecting electrode, the first potential-measuring electrode, the second current-injecting electrode and/or the second potential-measuring electrode may be of different types when they are integrated into the circular sleeve. In particular, electrodes that operate in a “dry” environment may be chosen. “Dry” is understood to mean that said electrodes do not require the use of a conductive gel. A conductive gel, usually used for electrodes that are positioned on a user as a one-off, is not suitable for continuous wearing of the electrodes. Specifically, the gel would itself also have to be worn for a long time, which could irritate the skin. Moreover, these electrodes are compatible with operation in a wet environment. Specifically, they should be able to withstand the perspiration of the user and to operate there, especially since the close contact between the band and the user's wrist may cause sweating.

According to one aspect of the invention, the cylindrical sleeve incorporates circular electrodes. These are the first current-injecting electrode, the first potential-measuring electrode, the second current-injecting electrode and/or the second potential-measuring electrode. These circular electrodes define a path that is parallel overall to the edges of the cylindrical sleeve, thus forming a closed circle. The elastic cylindrical sleeve and the circular electrodes are intimately linked. This configuration promotes contact between the electrodes and the wrist. The electrodes, just like the cylindrical sleeve, are thus ergonomic. In other words, the combination of an elastic cylindrical sleeve and one or more circular electrodes makes it possible to have a device that is easy to insert around the wrist for the user and loose enough so as not to impair the user, while still allowing the measurement to be reliable by ensuring that at least part of the body of the user is in contact with the corresponding circular electrode.

These dry electrodes, which are integrated into the cylindrical sleeve, are thus circular in a particular way. The cylindrical sleeve and the circular electrode may correspond to a weave covered with a conductive element, such as metal, more particularly silver. They may also correspond to a weave covered with a conductive reinforced polymer, such as a polysiloxane polymer.

These weaves may have a solid surface or a structured surface. The structured surfaces may be a porous surface or a ribbed surface. In a wet context, the favorable combination for an electrode/band pair will be: structured surface and electrode formed of a polysiloxane polymer.

According to one aspect of the invention, the current-injecting electrode and the potential-measuring electrode are of different dimensions. In order to optimize the measurement, the potential-measuring electrode adopts specific dimensions, and in particular a surface area larger than that of the corresponding injecting electrode, that is to say the one arranged on the same face as the measuring electrode. The dimensions of the electrodes should make it possible to ensure a contact interface with the user, and the large surface area of the measuring electrode should make it possible to reduce the stray impedances of these electrodes.

An inner contact surface corresponds to the inner surface of the band, including a surface of the first potential-measuring electrode intended to be in contact with the wrist of the first arm of the user. This surface of the first potential-measuring electrode intended to be in contact with the wrist is a first interface surface.

An outer contact surface corresponds to the outer surface of the band, including a surface of the second potential-measuring electrode intended to be in contact with the hand part of the user. This surface of the second potential-measuring electrode intended to be in contact with the hand part of the user is a second interface surface.

The first interface surface and the second interface surface have a specific geometry. The first interface surface and the second interface surface of the measuring electrodes is thus wide. The wider they are, the better the measurement. A first interface surface, respectively a second interface surface, should be understood to be wide when it represents a surface area greater than at least 10% of the inner surface, respectively outer surface of the band, and advantageously a surface area greater than 40% of the inner or outer surface of the band.

As an alternative, the second interface surface is configured such that the hand part of the second arm has the shape of at least one finger. In order to close the current flow loop, the user thus pauses his finger on the potential-measuring electrode. In this configuration, another finger comes into contact with the second current-injecting electrode.

As another alternative, the second interface surface is configured such that the hand part of the second arm has the shape of a palm. In order to close the current flow loop, the user thus grasps the measuring device. In this configuration, the second current-injecting electrode, close to the second potential-measuring electrode, is enclosed by the palm and thus comes into contact with the hand of the second arm of the user.

According to one aspect of the invention, the band comprises at least one positioning marker for positioning the part of the second arm of the user. For the sake of reproducibility of the measurement, the user is assisted by positioning markers. These positioning markers are coincident with or incorporate the second current-injecting electrode and the second potential-measuring electrode, on the outer surface of the band. The contact geometry, which is known to impact the measurement, is thus not modified by postural variability of the user.

According to one embodiment, the positioning marker is a contour of the contact surface. This contour is for example shown on the band by a picture, or by any other means. It may show one or more fingers, or the imprint of the hand, in full or in part.

According to another embodiment, the positioning marker is signified by an elevation at the position of the second current-injecting electrode and of the second potential-measuring electrode. This elevation may constitute the contour of the contact surface. The elevation may be limited to notches arranged along the contour of the contact surface.

According to another embodiment, the positioning marker is a depressed surface. The positioning marker is thus set back from a main plane of the outer surface of the band toward the inner surface of the band, thus reducing the distance between the outer surface and the inner surface of the band. Conversely, the positioning marker is a protruding surface.

According to one aspect of the invention, the measuring device comprises an iterative measurement alarm. The current flow loop is closed through contact with part of the second hand of the user. This flow loop is closed at the initiative of the user. However, a monitoring protocol requires regular measurements.

The iterative alarm is a recurring time alarm. It tells the user that the measurement should be taken. According to one particular protocol, the measurements are spaced apart by two hours. The iterative alarm may take the form of a visual, acoustic or tactile alert. Without limitation, mention may be made of: an indicator light, or a particular display on a user interface, a ringtone, a vibration of the measuring device, or a combination of these examples. The iterative alarm may be interrupted manually, or automatically when the new monitoring measurement is taken.

The iterative alarm may be combined with the one-off alert system, warning that the measurement has not been taken correctly, to form the same alarm. They may thus be of the same nature. As an alternative, the iterative alarm is separate from the one-off alert.

Taking regular measurements is beneficial in terms of analyzing the raw data, obtaining their average corresponding to the monitoring measurement, and comparing these monitoring measurements with a benchmark value. Thus, in the case of calculating a daily average measurement, having the same amount of raw data to be averaged and/or the fact that these measurements were taken at equivalent times from the point of view of the user's biological rhythms and in particular circadian rhythm, guarantees the reproducibility of the measurements. As a result, any deviation in the monitoring measurements, which deviation is observed in the proportions as explained above, will signify a pulmonary anomaly, and not an artefact due to the user's biological rhythm.

The invention also relates to a method for determining information relating to a transthoracic bioimpedance, comprising the following steps:

Wearing, on the wrist of the first arm of a user, a measuring device as claimed in any one of the preceding claims;

Positioning part of the second arm of the user on the measuring device in order to carry out a measurement;

Emitting a current so that said current flows between two current-injecting electrodes, connected by the loop formed by the contact between the measuring device and, respectively, the first arm wearing the measuring device and the second arm positioned on this measuring device, the current imposed by the injecting electrodes passing through a flow loop via a transthoracic segment of the user, the first injecting electrode being arranged on the inner surface of the band, the second injecting electrode being arranged on the outer surface of the band, and two potential-measuring electrodes arranged in the flow loop, the first measuring electrode being arranged on the inner surface of the band and the second measuring electrode being arranged on the outer surface of the band;

Measuring a potential difference between the two measuring electrodes and obtaining raw data;

Transferring the raw data to the processing system;

Formulating a monitoring measurement resulting from an average of a plurality of raw data and comparing this monitoring measurement with a reference benchmark value:

Obtaining information relating to a transthoracic bioimpedance;

Transmitting the information to the user via a user interface or to a medical representative, such as a doctor or an emergency center.

According to one feature of the invention, the value of each item of raw data may also be compared with recorded threshold values, such as a limit value. The user may then be informed of the validity of the monitoring measurement.

According to the invention, the user is warned of a significant variation possibly indicative of heart failure when the step of comparing the monitoring measurement, that is to say an averaged value of a plurality of raw data, with the reference benchmark value identifies a deviation of the order of 0.25 to 2.5% from the benchmark value.

### BRIEF DESCRIPTION OF THE DRAWINGS

Other features, details and advantages of the invention will become more clearly apparent on reading the description given below by way of indication with reference to the drawings, in which:

FIG. 1 is a general view of the context of use of the measuring device according to the invention,

FIG. 2 is a flowchart of the operation of a measuring device according to the invention,

FIG. 3 is a schematic view of a measuring device according to the invention in a first embodiment,

FIG. 4 is a perspective view of a measuring device according to the invention in a second embodiment.

### DETAILED DESCRIPTION OF THE INVENTION

It should first of all be noted that the figures set out the invention in detail in order to implement the invention, said figures of course being able to serve to better define the invention if necessary.

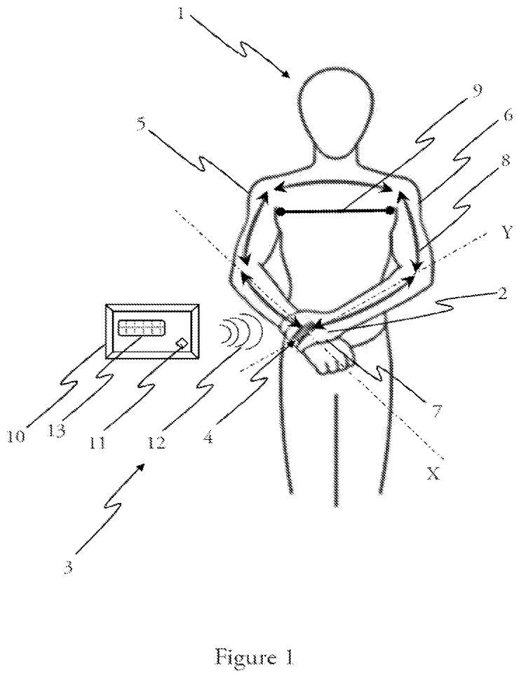

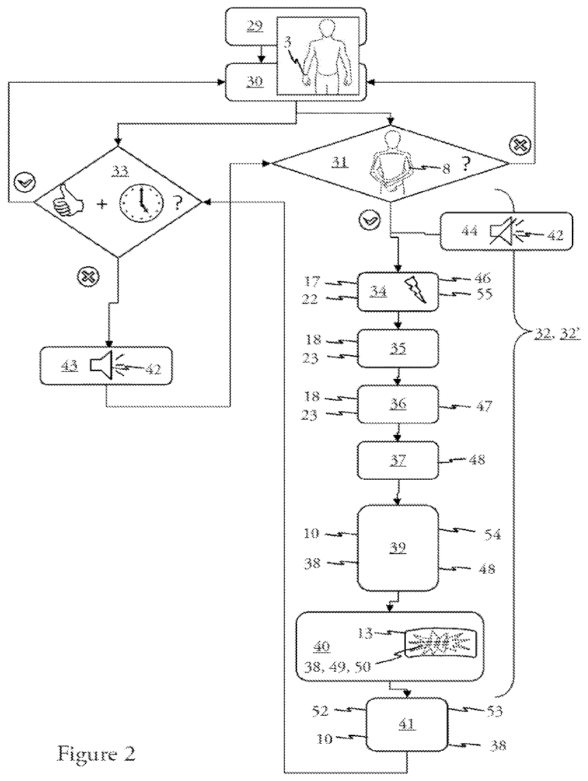

With reference first of all to FIGS. 1 and 2, the operation of the measuring device and the associated measuring method will be described. FIG. 1 shows a user 1 wearing, on a wrist 2, a bioimpedance-measuring device 3 according to the invention. The measuring device 3 takes the form of a band 4, seen here transparently through a hand of the user, the band 4 surrounding the wrist 2 of a first arm 5 of the user 1, in this case the right arm.

The band 4 of the measuring device 3 has an inner surface 21 facing the wrist 2 when the band 4 is worn by the user 1, and an outer surface 19 facing away therefrom.

The inner surface 21 carries a first current-injecting electrode 22 and a first potential-measuring electrode 23. By being worn on the wrist 2 of the user 1, this first current-injecting electrode 22 and this first potential-measuring electrode 23 are able to come into contact with the wrist 2.

The outer surface 19 carries a second current-injecting electrode 17 and a second potential-measuring electrode 18. The second current-injecting electrode 17 and the second potential-measuring electrode 18 are each configured so as to have an interface surface 56 with part 7 of the second arm 6 of the user 1, here a palm and fingers of the hand of the left arm.

When the user 1 grasps the measuring device 3, the second current-injecting electrode 17 and the second potential-measuring electrode 18 come into contact with the left hand of the user 1. Through this movement, the user 1 closes a current flow loop 8.

The band 4 comprises an electric power supply system 46 delivering a current 55 able to flow between the current-injecting electrodes when the flow loop 8 is closed. In this path, forming the flow loop 8, the current 55 passes through the first arm 5 as well as the second arm 6 of the user 1 and, between them, a transthoracic segment 9.

The current 55 then flows from the first current-injecting electrode 22 to the second current-injecting electrode 17, passing successively through the first potential-measuring electrode 23 and the second potential-measuring electrode 18.

When the current 55 flows in the flow loop 8 through the body of the user, the electrical potential value that results therefrom is dependent on the resistance encountered on the current path, this resistance varying in particular depending on the water content of the body parts passed through. A specific signal is thus delivered to the first potential-measuring electrode 23 and to the second potential-measuring electrode 18. This signal corresponds to raw bioimpedance data 48. The first potential-measuring electrode 23 and the second potential-measuring electrode 18 transmit these raw data 48 to a collection system 47 arranged on the band 4.

The collection system 47 is configured so as to communicate with a processing system 10. In the embodiment shown in FIG. 1, the processing system 10 is externalized with respect to the band 4. The collection system 47 transmits the raw data 48, via a first communication peripheral, to the processing system 10 carrying a second communication peripheral 11. The first communication peripheral and the second communication peripheral 11 communicate here by propagating the signal in the form of waves 12.

The processing system 10 applies a prerecorded algorithm to the raw data 48. According to one embodiment, the algorithm is configured so as to take an average of several consecutive raw data 48 measurements corresponding to a monitoring measurement 32, 32′ in order to obtain a result 38 that will then be compared with the floor and limit threshold values previously defined and stored in the processing system 10. The raw data 48 are thus processed in order to obtain information 50 corresponding to the comparison between the monitoring measurement 32 implementing the average of the raw data 48 and a benchmark value 52.

The information 50 is then presented to the user 1 via a user interface 13. This user interface 13 is carried by the processing system 10 in the illustrated example. The user interface 13 may make it possible to display various data, for example the information 50, the deviation 53 between the monitoring measurement 32 and the benchmark value 52, the need to perform a new monitoring measurement 32′, etc.

FIG. 2 shows a flowchart representative of an implementation of a bioimpedance-measuring device 3 according to the invention for obtaining a monitoring measurement 32.

When a user, in a step 29, activates the measuring device 3 worn on the wrist of a first arm, this measuring device is in the standby state 30. When the user forms a flow loop 8 by touching the measuring device 3 with a second arm, a monitoring measurement 32 is performed. This monitoring measurement 32 corresponds to steps 31 to 41.

The flow loop 8 is formed through a positive action by the user that creates contact between the measuring device and a first arm, via the inner surface of the band, and a second arm, via the outer surface of the band. This contact is identified by the measuring device 3 in a step 31. In a step 34, an electric power supply system 46 is driven so as to generate a current supply 55 between a first current-injecting electrode 22 and a second current-injecting electrode 17. The current 55 then flows between these two injecting electrodes in the current flow loop 8 through the body, the band also providing electrical insulation between the two injecting electrodes, which forces the current to pass through the body, successively through the first arm, a transthoracic segment and the second arm. On the current path, the current 55 emitted between each injecting electrode 17, 22 passes through the measuring electrodes 23, 18 arranged on the path, such that, in a measuring step 35, an electrical potential measurement is performed at the first measuring electrode 23 and an electrical potential measurement is performed at the second measuring electrode 18.

The electrical potential value is transferred, in a transfer step 36, from each of the measuring electrodes to a collection system 47 which, in a transmission step 37, transmits them to a processing system 10 configured so as to calculate the potential difference corresponding to the “raw data” 48.

The processing system 10, in a calculation step 39, processes the raw data 48. Firstly, each item of raw data 48 obtained, corresponding to a potential difference measurement, is compared with a recorded limit value 54, attesting to the reliability of the measurement. This limit value 54 may for example represent 150% of the reference benchmark value 52, which will be introduced below. At item of raw data 48 with a value greater than this limit value 54 implies an impedance value that is far too high and the risk of an unreliable measurement, for example due to incorrect contact conditions between the measuring device and the body of the user. It is understood that, if such unreliable measurement information should be identified, the measurement process ends and should be restarted.

The processing system 10 then compiles the raw data obtained over a given period in order to obtain a result 38 corresponding to an average of these raw data 48, said average being indicative of reliable information on the bioimpedance measurement of the user for a given day.

The result 38 obtained may be transmitted to the user on a user interface 13, in a display step 40.

The result 38 is moreover evaluated in a test step 41 by the processing system 10. The processing system 10, in the test step 41, compares the result 38 with a reference benchmark value 52 resulting from a calibration method and previously carried out and stored in a memory of the processing system 10.

The averages are calculated and stored in the memory of the processing system 10, so that the presence of an unfavorable evolution in the user's condition is identified. The result of this comparison is considered with regard to a floor value specific to the present invention. If a deviation 53 of the order of 0.25 to 2.5% is identified, with the result 38 that is less than the reference benchmark value 52, the user is warned of this on the user interface 13.

After these various successive steps, the measuring device 3 returns to the standby state 30. It remains there for a determined duration, for example two hours following a valid monitoring measurement 32, unless the user closes the current flow loop 8 beforehand. Beyond two hours after carrying out a monitoring measurement 32, the exceedance of which is monitored in step 33, an alarm 42 is triggered automatically in a step 43, iteratively, in order to warn the user of this and encourage him to perform a new monitoring measurement 32′. The alarm 42 is interrupted by a step 44 when the current flow loop 8 is formed again.

If the monitoring measurement 32 should be considered invalid, the measuring device is able to activate the alarm 42 in order to warn the user of this, inviting said user to reproduce the monitoring measurement 32.

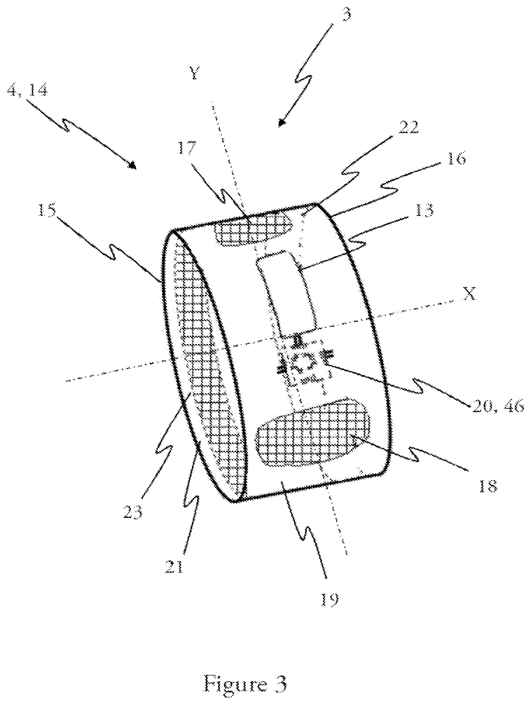

FIG. 3 shows a bioimpedance-measuring device 3 comprising a band 4 as shown above. This band 4 in this case takes the form of an elastic cylindrical sleeve 14. The elastic cylindrical sleeve 14 extends along an axis of elongation X.

The elastic cylindrical sleeve 14 is provided with edges 15, 16 that define the ends of the sleeve along the axis of elongation X. The edge 15 is in this case called the proximal edge in that it is the edge facing the transthoracic segment of the user, whereas the edge 16 is in this case called the distal edge in that it faces away from the transthoracic segment, closest to the hand of the arm wearing the measuring device.

The elastic cylindrical sleeve 14 does not have any interruptions over its circumference, such that circular electrodes are able to be placed there, extending over the entire circumference of the elastic cylindrical sleeve 14, around the axis of elongation X of the sleeve. A first current-injecting electrode 22, forming an uninterrupted circle and shown here only in dotted lines, and a first potential-measuring electrode 23, forming an uninterrupted circle, are arranged on the inner surface 21 of the band 4 formed by the elastic cylindrical sleeve 14. It should be noted that, with these two electrodes being arranged on the inner surface 21 of the band 4, the user is not able to ensure that his body, here his wrist, is correctly in contact with the electrodes. Implementing a circular electrode makes it possible to ensure contact between the electrode and the wrist regardless of the pressure exerted by the hand closing the flow loop.

Moreover, a second current-injecting electrode 17, in this case forming a substantially circular spot, and a second potential-measuring electrode 18, in this case forming a substantially elliptical spot, are arranged on the outer surface 19 of the band 4 formed by the elastic cylindrical sleeve 14.

The first electrodes arranged on the inner face are distinguished by their position on the first arm 5 wearing the measuring device 1, with one electrode close to the proximal edge 15 and one electrode close to the distal edge 16. According to the invention, the electrode close to the proximal edge is the first potential-measuring electrode 23 and the electrode close to the distal edge is the first injecting electrode 22. The first injecting electrode 22 is thus the closest to the hand of the first arm 5 wearing the measuring device 1, and the first measuring electrode 23 is in the path of the current leaving the first injecting electrode 22.

The first current-injecting electrode 22 and the first potential-measuring electrode 23 are of the same dimensions in this case. They are arranged parallel to each other, forming a first group of electrodes in which the electrodes are aligned in a first direction X corresponding to the direction of elongation of the first arm. The electrodes are moreover also parallel to the edges 15, 16 of the elastic cylindrical sleeve 14.

The second electrodes arranged on the outer surface 19 are distinguished by their position with respect to the hand of the user resting on the measuring device, with the second injecting electrode 17 arranged so as to be in contact with the fingers of this hand and the second measuring electrode 18 arranged so as to be in contact with the palm of this hand.

The second current-injecting electrode 17 and the second potential-measuring electrode 18 have different dimensions in this case, it being notable that the second potential-measuring electrode 18 is wider than the second current-injecting electrode 17. This difference in dimensions is justified by a desire to increase the surface area of the measuring electrodes as much as possible, so that the stray impedance of the electrodes is low in comparison with the bioimpedance of the body passed through by the current, whereas the definition of the width of the injecting electrode relates only to the need for effective contact. It should be noted that this difference in dimensions has the advantage of dedicating the second measuring electrode 18, which is wider than the second injecting electrode 17, to an area in which the palm of the user should be pressed, enabling this large contact surface between the body of the user and the measuring electrode, whereas the second injecting electrode 17 is dedicated to positioning the fingers of the user.

The second electrodes form a second group of electrodes in which the second electrodes are aligned in a second direction Y corresponding to the direction of elongation of the second arm, or in other words in a direction perpendicular to the direction of elongation of the arm wearing the measuring device. As may be seen in FIG. 3 in particular, the first electrodes are arranged in series in a first direction that is perpendicular or substantially perpendicular to the second direction in which the series formed by the second electrodes extends.

The elastic cylindrical sleeve 14 consists of a weave covered with polysiloxane polymers forming the first current-injecting electrode 22 and the first potential-measuring electrode 23. The first current-injecting electrode 22 and the first potential-measuring electrode 23 have a surface structured with ribs.

The outer surface 19 of the elastic cylindrical sleeve 14 also carries a user interface 13. This user interface 13 takes the form of an electronic display device, for example a liquid-crystal screen or light-emitting diode screen.

An electric power supply system, a collection system and a processing system are supported by a printed circuit board 20, made visible transparently here so as to allow it to be identified by the reader. This printed circuit board 20 is integrated into the elastic cylindrical sleeve 14. It forms an electric power supply system 46 as has been explained above, which has a wired connection to the first current-injecting electrode 22 and the first potential-measuring electrode 23 arranged on the inner surface 21 of the elastic cylindrical sleeve 14, and to the second current-injecting electrode 17 and the second potential-measuring electrode 18 arranged on the outer surface 19, as well as to the user interface 13.

The user interface 13 and the printed circuit board 20 are located between the first current-injecting electrode 22 and the first potential-measuring electrode 23. This geometry makes it possible to comply with a spacing between the electrodes, thus avoiding interference. The geometry is also such that the presence of the user interface 13 generates a spacing between the electrodes arranged on the outer face of the elastic cylindrical sleeve 14, so as to avoid interference and to generate an adequate spacing for simultaneous contact of the palm and the fingers on their respective electrode.

The cylindrical sleeve 14 has elastic properties, allowing it to adapt to the dimensions of a user's wrist. For greater comfort of use, the printed circuit 20 will be flexible in order to adapt to an elastic cylindrical sleeve 14. For example, a flex circuit, also known by the name flex PCB, may be used. In the same way, the user interface 13 may be flexible. Failing that, the user interface 13 will preferably be profiled so as to follow a rounding in order to conform to the wrist.

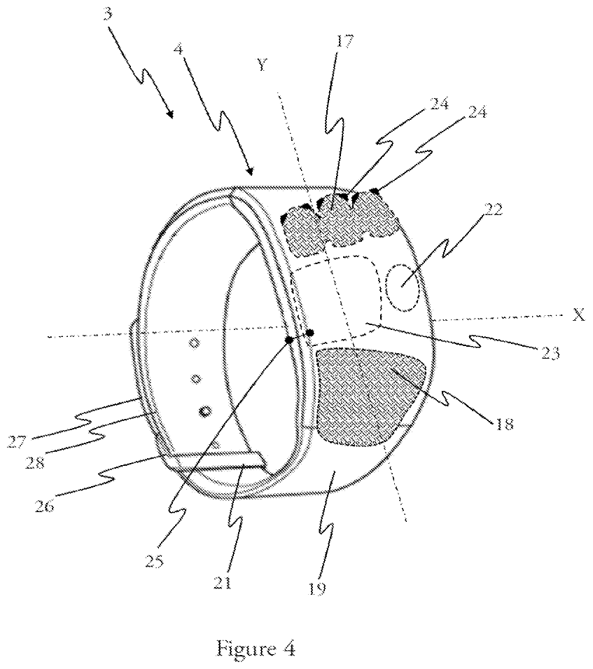

FIG. 4 shows another embodiment of a bioimpedance-measuring device 3. The band 4 is provided with a clasp 26 for making overlapping ends 27, 28 interact. When the ends 27, 28 interact, the band 4 forms a closed cylinder able to be placed around the arm of the user. The clasp 26 is in this case a pin-buckle clasp, the band 4 therefore being adjustable to the dimensions of the wrist of each user.

The band 4 incorporates non-circular electrodes 17, 18, 22, 23, arranged locally on the band 4. In particular, a first current-injecting electrode 22 and a first potential-measuring electrode 23 are of different dimensions. More particularly, the first potential-measuring electrode 23, illustrated transparently, covers a surface area larger than the first current-injecting electrode 22, also illustrated transparently, in order to make the stray impedance of this measuring electrode low, as explained above. The same applies for a second current-injecting electrode 17 and a second potential-measuring electrode 18. In this exemplary embodiment, the first potential-measuring electrode 23 is of a size substantially equivalent to the second potential-measuring electrode 18.

In accordance with what has been described for the first embodiment, the electrode proximal to the transthoracic segment is the first potential-measuring electrode 23, whereas the electrode distal to the transthoracic segment is the first injecting electrode 22. The first injecting electrode 22 is thus closest to the hand of the first arm 5 wearing the measuring device 1. The first electrodes are arranged in series in a first direction X.

On the outer surface 19, intended to come into contact with in this case the hand of the second arm of the user, the electrode proximal to the transthoracic segment is the second potential-measuring electrode 18, whereas the electrode distal to the transthoracic segment is the second injecting electrode 17. The first electrodes are arranged in series in a second direction Y, substantially perpendicular to the first direction X.

The combination of these features relating to the distal/proximal positioning of the injecting and measuring electrodes is notable in this case in that the current-injecting electrodes are positioned distally on each surface of the measuring device, such that implementation of the largest possible flow loop from one injecting electrode to the other is ensured.

The electrodes carried by an inner surface 21 of the band, that is to say the first current-injecting electrode 22 and the first potential-measuring electrode 23, are elliptical in shape. The electrodes carried by an outer surface 19 of the band, that is to say the second current-injecting electrode 17 and the second potential-measuring electrode 18, take the form of the end of a finger corresponding to the distal phalanges. These are provided with positioning markers 24 corresponding to projections, for example tabs, on which a user will have to bring together two distal phalanges of his second arm. The measurement reproducibility inherent to a contact geometry will thus be ensured.

It is notable according to this embodiment that all of the electrodes, both the first electrodes arranged on the inner surface of the band and the second electrodes arranged on the outer surface of the band, are arranged in a front part of this band, that is to say the part of the band opposite the clasp. It is thereby ensured, when the user presses on the band with his free hand, that the first electrodes will be firmly pressed against the first arm wearing the measuring device.

As has been described above, the band 4 is in communication with an externalized processing system. A signal collected by the collection system 47 embedded in the band 4 and communicating with each of the potential-measuring electrodes 23, 18 is thus transmitted. This signal thus comes both from the first potential-measuring electrode 23 and from the second potential-measuring electrode 18, and corresponds to raw impedance data 48. The collection system 47 is provided with a first communication peripheral. This collection system 47 is arranged in a thickness 25 of the band 4. The first communication peripheral is configured so as to communicate with the processing system 10 carrying a second communication peripheral 11. The thickness 25 of the band 4 also contains the electric power supply system 46 connected to the four electrodes 17, 18, 22, 23.

It will be understood from reading the above that the present invention proposes a bioimpedance-measuring device configured so as to achieve a gain in terms of compactness, practicality and ease of use in comparison with existing devices. This bioimpedance-measuring device is intended to be worn continuously by a user who, through a simple action, performs a monitoring measurement in order to monitor the evolution of his lung condition. Such a bioimpedance-measuring device, using a large Piccoli vector, also achieves a gain in terms of accuracy in comparison with the prior art.

The invention should not however be limited to the means and configurations that are described and illustrated here, and it also extends to any equivalent means or configuration and to any technical combination using such means. In particular, the form of the bioimpedance-measuring device may be modified without impacting the invention, insofar as the bioimpedance-measuring device ultimately performs the same functions as those described in this document.

1-13. (canceled)

14. A bioimpedance-measuring device, comprising:
a band configured so as to surround a wrist of a first arm of a user and on which there are arranged at least two current-injecting electrodes and at least two potential-measuring electrodes;
a collection system for collecting raw bioimpedance data from the at least two potential-measuring electrodes; and
a raw data processing system for obtaining, based at least on the collected raw bioimpedance data, at least one monitoring measurement and information signifying a pulmonary bioimpedance condition, wherein
a first current-injecting electrode of the at least two current-injecting electrodes and a first potential-measuring electrode of the at least two potential-measuring electrodes are arranged on an inner surface of the band so as to be in contact with the wrist of the first arm of the user,
a second current-injecting electrode of the at least two current-injecting electrodes and a second potential-measuring electrode of the at least two potential-measuring electrodes are arranged on an outer surface of the band and configured so as to have an interface surface with a part of a second arm of the user, and
the collection system is carried by the band and configured so as to communicate with the raw data processing system.

15. The bioimpedance-measuring device according to claim 14, wherein
the first current-injecting electrode of the at least two current-injecting electrodes and the first potential-measuring electrode of the at least two potential-measuring electrodes arranged on the inner surface of the band define a first group of electrodes,
the second current-injecting electrode of the at least two current-injecting electrodes and the second potential-measuring electrode of the at least two potential-measuring electrodes arranged on the outer surface of the band define a second group of electrodes, and
electrodes of the first group of electrodes are aligned in a direction different from a direction in which electrodes of the second group of electrodes are aligned.

16. The bioimpedance-measuring device according to claim 14, wherein
on each surface of the band, an electrode proximal to a transthoracic segment of the user is a respective potential-measuring electrode and an electrode distal to the transthoracic segment of the user is a respective current-injecting electrode.

17. The bioimpedance-measuring device according to claim 14, wherein
the first current-injecting electrode of the at least two current-injecting electrodes and the first potential-measuring electrode of the at least two potential-measuring electrodes arranged on the inner surface of the band define a first group of electrodes, the first group of electrodes being aligned in a first direction along a longitudinal axis of the first arm of the user, the first current-injecting electrode of the first group of electrodes being arranged on one of a medial aspect or a lateral aspect of the wrist of the first arm of the user and the first potential-measuring electrode of the first group of electrodes being arranged on the other one of the medial aspect or the lateral aspect of the wrist of the first arm of the user.

18. The bioimpedance-measuring device according to claim 14, wherein the raw data processing system is configured so as to
determine a deviation between a reference benchmark value and the obtained at least one monitoring measurement and to generate a notification for the user when
the obtained at least one monitoring measurement has a value less than the reference benchmark value, and
the determined deviation is between 0.25% and 2.5% of the reference benchmark value.

19. The bioimpedance-measuring device according to claim 14, wherein the raw data processing system compares the collected raw bioimpedance data with at least one limit value.

20. The bioimpedance-measuring device according to claim 14, wherein the raw data processing system is integrated into the band.

21. The bioimpedance-measuring device according to claim 14, wherein the at least two current-injecting electrodes are driven so as to use a multi-frequency current.

22. The bioimpedance-measuring device according to claim 14, wherein the band is formed by an elastic cylindrical sleeve.

23. The bioimpedance-measuring device according to claim 22, wherein the elastic cylindrical sleeve incorporates, as at least a portion of the at least two current-injecting electrodes and the at least two potential-measuring electrodes, circular electrodes.

24. The bioimpedance-measuring device according to claim 14, wherein the at least two current-injecting electrodes and the at least two potential-measuring electrodes are of different dimensions.

25. The bioimpedance-measuring device according to claim 14, wherein the band comprises at least one positioning marker for positioning the part of the second arm of the user.

26. A method for determining information relating to a transthoracic bioimpedance, the method comprising:
wearing, on a wrist of a first arm of a user, a bioimpedance-measuring device;
positioning a part of a second arm of the user on the bioimpedance-measuring device in order to carry out a measurement;
emitting, by processing circuitry, a current flowing between at least two current-injecting electrodes, connected by a flow loop formed by contact between the bioimpedance-measuring device and, respectively, the first arm of the user wearing the bioimpedance-measuring device and the second arm of the user positioned on the bioimpedance-measuring device, the current imposed by the at least two current-injecting electrodes passing through the flow loop via a transthoracic segment of the user, a first current-injecting electrode of the at least two current-injecting electrodes being arranged on an inner surface of a band of the bioimpedance-measuring device, a second current-injecting electrode of the at least two current-injecting electrodes being arranged on an outer surface of the band of the bioimpedance-measuring device, and at least two potential-measuring electrodes arranged in the flow loop, a first potential-measuring electrode of the at least two potential-measuring electrodes being arranged on the inner surface of the band of the bioimpedance-measuring device and a second potential-measuring electrode of the at least two potential-measuring electrodes being arranged on the outer surface of the band of the bioimpedance-measuring device;
obtaining, by the processing circuitry, raw bioimpedance data corresponding to a potential difference between the at least two potential-measuring electrodes;
formulating, by the processing circuitry and based on the obtained raw bioimpedance data, a monitoring measurement by averaging a plurality of the obtained raw bioimpedance data;
comparing, by the processing circuitry, the formulated monitoring measurement with a reference benchmark value;
obtaining, by the processing circuitry and based on the comparing, information relating to the transthoracic bioimpedance; and
transmitting, by the processing circuitry, the obtained information to one of the user via a user interface or to a medical representative.
